(19) United States
(12) Plant Patent Application Publication
(10) Pub. No.: US 2025/0008853 P1
(43) Pub. Date: Jan. 2, 2025

(54) LEMON PLANT NAMED 'CE-D5-1-9-42'

(50) ;

(71) Applicant: Florida Foundation Seed Producers, Inc., Marianna, FL (US)

(72) Inventor: Frederick GMITTER, Jr., Lakeland, FL (US)

(21) Appl. No.: 18/751,529

(22) Filed: Jun. 24, 2024

Related U.S. Application Data
(60) Provisional application No. 63/510,537, filed on Jun. 27, 2023.

(51) Int. Cl. A01H 6/78

(52) U.S. Cl. CPC A01H 6/785
USPC PLT/201

(57) ABSTRACT

A new and distinct cultivar of lemon plant named ‘CE-D5-1-9-42’, characterized by nearly seedless fruit, early fruiting, and high fruit production. Additionally, the new lemon cultivar also is compatible with trifoliate orange rootstocks, unlike most lemon varieties. This new cultivar has high quality fresh fruit, as well as juice, peel oil, and other lemon by-products viable for processing. Further, given the new lemon cultivar's good HLB tolerance typical of lemons, and its near seedlessness, this new cultivar may have significant dooryard tree potential.

### CROSS-REFERENCE TO RELATED APPLCIATIONS

This Application claims the benefit of U.S. Provisional Patent Application No. 63/510,537, filed Jun. 27, 2023, which is incorporated by reference herein in its entirety.

### ACKNOWLEDGEMENT OF FEDERAL RESEARCH SUPPORT

N/A.

Genus and species: Citrus limon.

Cultivar denomination: ‘CE-D5-1-9-42’.

### BACKGROUND OF THE NEW CULTIVAR

The present invention relates to a new and distinct cultivar of lemon, botanically known as Citrus sp. and hereinafter referred to by the name ‘CE-D5-1-9-42’.

The new lemon cultivar ‘CE-D5-1-9-42’ arose as an irradiated budwood clone from ‘Cook Eureka’ lemon (not patented), produced as part of a large project to generate lemon clones that could produce higher yields of good quality peel oil for industrial use. The original tree was planted in Fort Pierce in 1999. The new cultivar ‘CE-D5-1-9-42’ was among a second group of clones selected for high oil production and was included in a large field trial planted in South America under a materials transfer agreement (MTA) with a local farm in 2013. Pathogen-tested budwood from FDACS-DPI was sent there, where it went through quarantine and further testing, for propagation onto multiple rootstocks including ‘Flying Dragon’ (not patented). Among 11 clones identified in the trial to be nearly seedless, higher young-tree production was observed on trees of ‘CE-D5-1-9-42’ on ‘Flying Dragon’ rootstock than with the other clones. Trueness-to-type was demonstrated by additional trees propagated on Afghan sour orange rootstock (not patented) and experimental rootstock hybrid ‘46x20-04-6’ (not patented) and planted in Fellsmere, Florida.

Plant Breeder's Rights for this cultivar have not been applied for. The new lemon cultivar ‘CE-D5-1-9-42’ has not been made publicly available more than one year prior to the priority filing date of this application.

### SUMMARY OF THE INVENTION

The new lemon cultivar ‘CE-D5-1-9-42’ has not been observed under all possible environmental conditions. The phenotype of the new cultivar may vary with variations in environment and cultural practices such as temperature, light intensity, fertilization, irrigation, and application of plant growth regulators without any change in genotype.

A new and distinct nearly seedless true lemon, which was originally selected for high peel oil production. The new cultivar ‘CE-D5-1-9-42’ was selected as a candidate for release based on its high early fruit production and near seedlessness. This cultivar can bring greater fresh fruit value to lemons grown in Florida, as well as good value in processing for juice, peel oil, and other lemon by-products. Further, with good citrus huanglongbing or greening (HLB) tolerance found in lemons, and its near seedlessness, this lemon cultivar may also have significant dooryard tree potential.

The new cultivar ‘CE-D5-1-9-42’ was identified as a seedless clone in the trial in Bolivia in 2017, where zero seed were found in 30 fruit. Subsequent seed counts show that most fruit are seedless, but a small percentage of fruit can have one or two small seeds. Fruit with more than two seeds have been observed, but this occurrence is very rare. This level of seedlessness is comparable to low-seeded lemon clones now finding favor in the domestic and international fresh fruit markets. Fruit appearance, fragrance, and juice content are typical of ‘Eureka’ lemon (not patented). Proprietary analyses of peel oil quality conducted in Tucuman, Argentina, concluded that the oils were commercially acceptable. Thus, this selection has good potential for fresh fruit or processed lemons grown in Florida, as well as for the door yard lemon tree market.

Most ‘Eureka’ lemon clones are incompatible with trifoliate orange rootstocks, so the good performance on ‘Flying Dragon’ (a trifoliate orange variety), with no indications of incompatibility, is surprising; if it holds true on other trifoliate orange rootstocks and hybrids, this would be a unique and quite valuable attribute. ‘Flying Dragon’ rootstock is widely used in the Tucuman lemon growing region for critical tree size control, but ‘Eureka’ lemons cannot be grown on ‘Flying Dragon’ rootstock there because of the incompatibility.

### DESCRIPTION OF THE FIGURES

The accompanying photographs (as shown in FIGS. 1-6B) illustrate the overall appearance of the new lemon cultivar ‘CE-D5-1-9-42’. These photographs show the colors as true as can be reasonably obtained in colored reproductions of this type. Colors in the photographs may differ slightly from the color values cited in the detailed botanical description, which accurately describe the colors of the new lemon cultivar.

FIG. 1 shows a three-year-old tree of the new lemon cultivar ‘CE-D5-1-9-42’ growing on ‘Flying Dragon’ rootstock in South America, with no HLB;

FIG. 2A shows a young tree of the new lemon cultivar bearing fruit of various sizes, growing in Florida in the spring season;

FIG. 2B shows the young tree of the new lemon cultivar of FIG. 2A, bearing fruit and newly opened flowers;

FIG. 2C shows clusters of fruit approaching maturity on a young potted tree of the new lemon cultivar;

FIG. 3A shows whole and cut mature fruit of the new lemon cultivar;

FIG. 3B shows whole and cut mature fruit of the new lemon cultivar;

FIG. 3C shows whole and cut mature fruit of the new lemon cultivar;

FIG. 3D shows whole and cut mature fruit of the new lemon cultivar;

FIG. 3E shows whole mature fruit of the new lemon cultivar;

FIG. 4A shows flower buds and flowers on a tree of the new lemon cultivar;

FIG. 4B shows flowers on a tree of the new lemon cultivar;

FIG. 4C shows flower buds on a tree of the new lemon cultivar;

FIG. 4D show flower buds and flowers on a tree of the new lemon cultivar;

FIG. 5A shows a flower and a flower bud of the new lemon cultivar alongside a ruler for measurement;

FIG. 5B shows flowers of the new lemon cultivar alongside a ruler for measurement;

FIG. 5C shows flowers of the new lemon cultivar alongside a ruler for measurement;

FIG. 5D shows flowers of the new lemon cultivar alongside a ruler for measurement;

FIG. 5E shows flowers of the new lemon cultivar alongside a ruler for measurement;

FIG. 5F shows flowers and flower buds of the new lemon cultivar in various stages of development alongside a ruler for measurement;

FIG. 5G shows flowers of the new lemon cultivar alongside a ruler for measurement;

FIG. 5H shows flowers of the new lemon cultivar with a ruler for measurement, with some petals and stamen having been removed from the flower shown on the righthand side of FIG. 5H to reveal the stigma, style, and ovary;

FIG. 5I shows a flower of the new lemon cultivar alongside a ruler for measurement;

FIG. 5J shows flowers of the new lemon cultivar alongside a ruler for measurement;

FIG. 6A shows fully expanded leaves of the new lemon cultivar alongside a ruler for measurement; and

FIG. 6B shows a fully expanded leaf of the new lemon cultivar alongside a ruler for measurement.

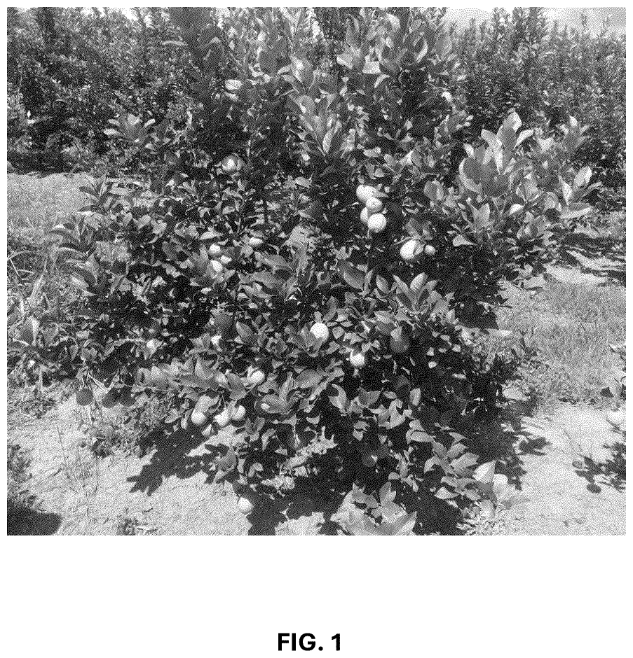

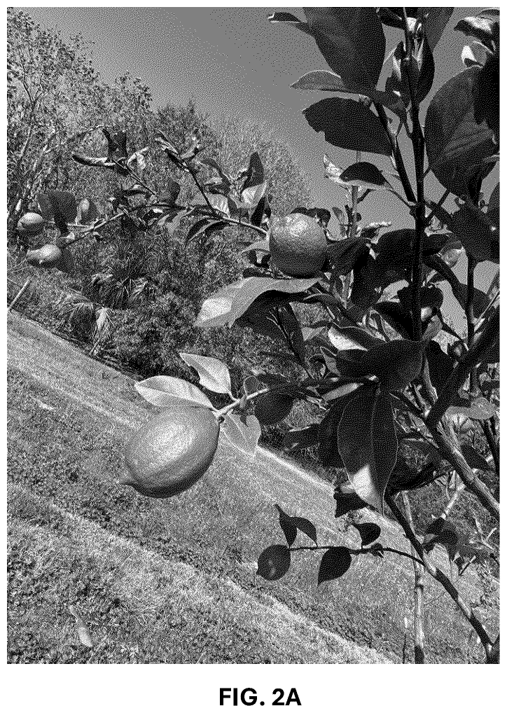

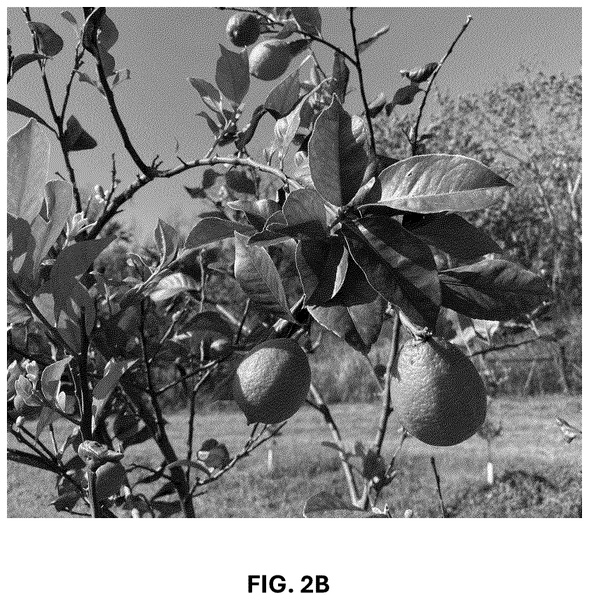

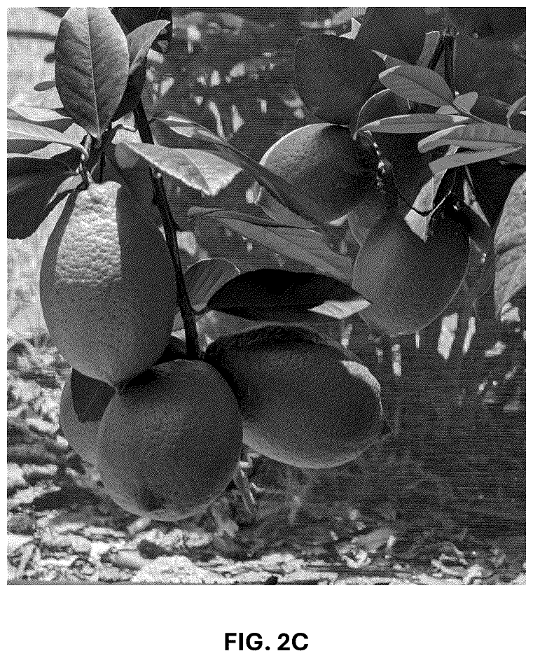

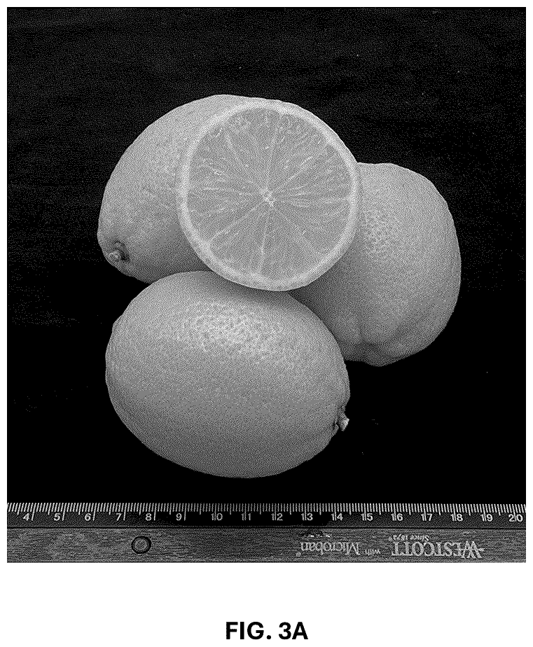

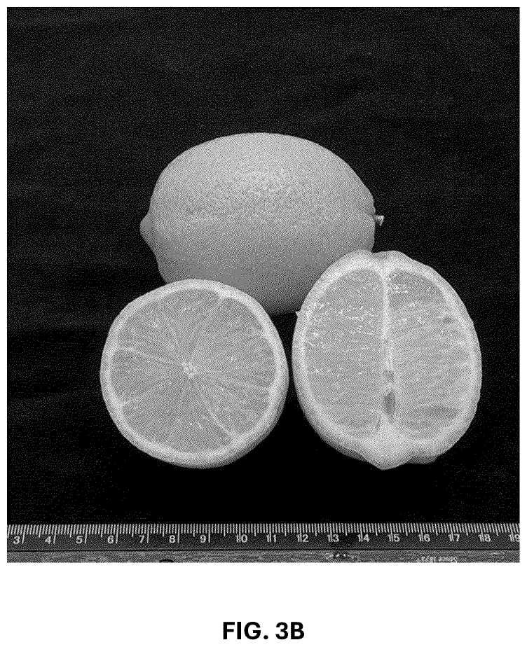

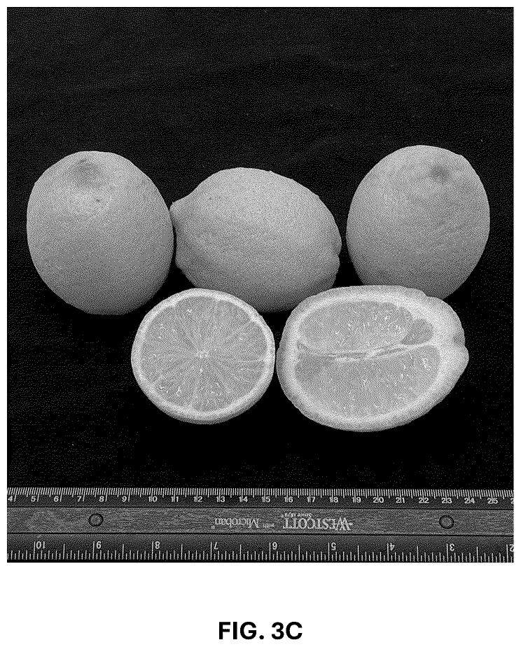

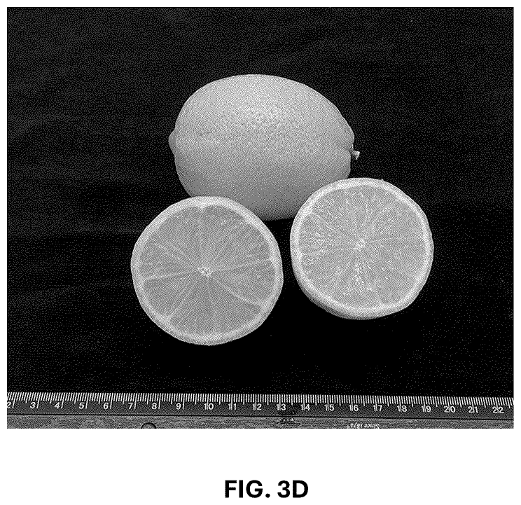

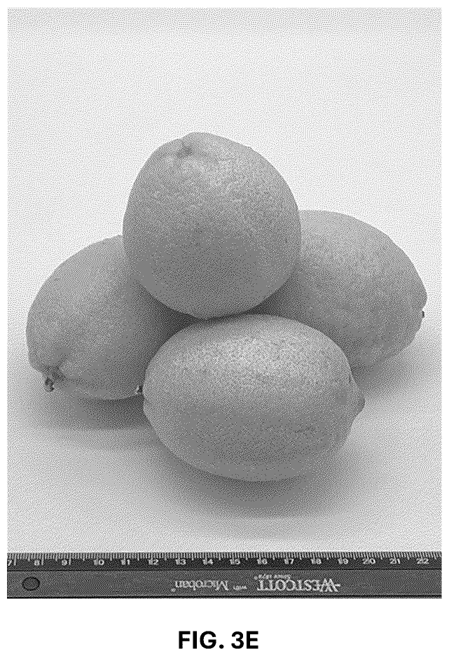

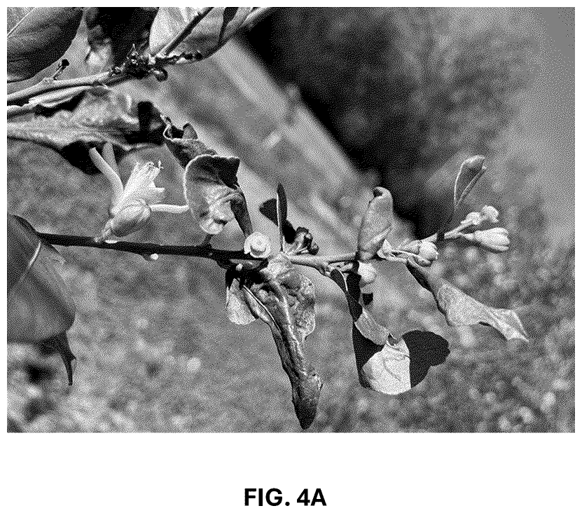

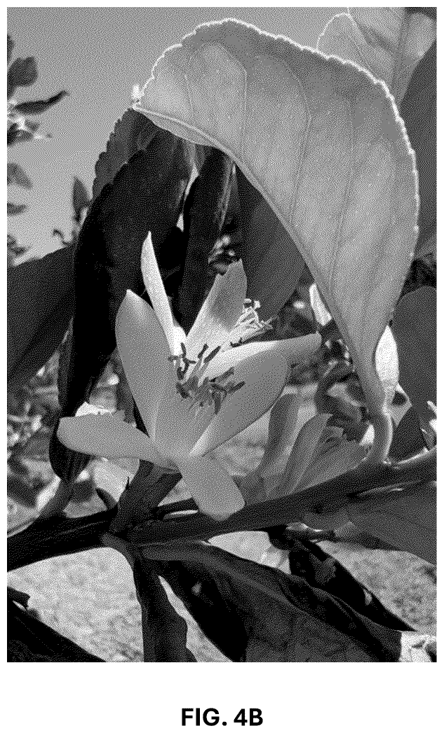

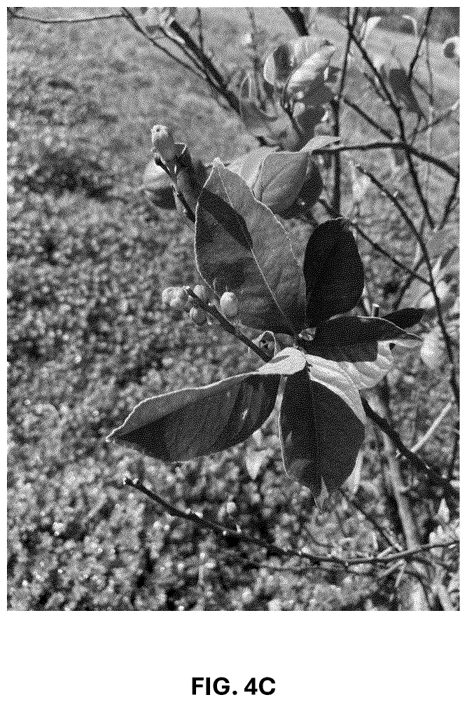

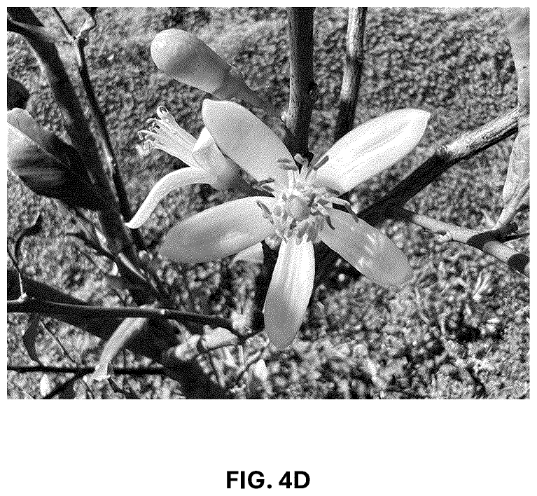

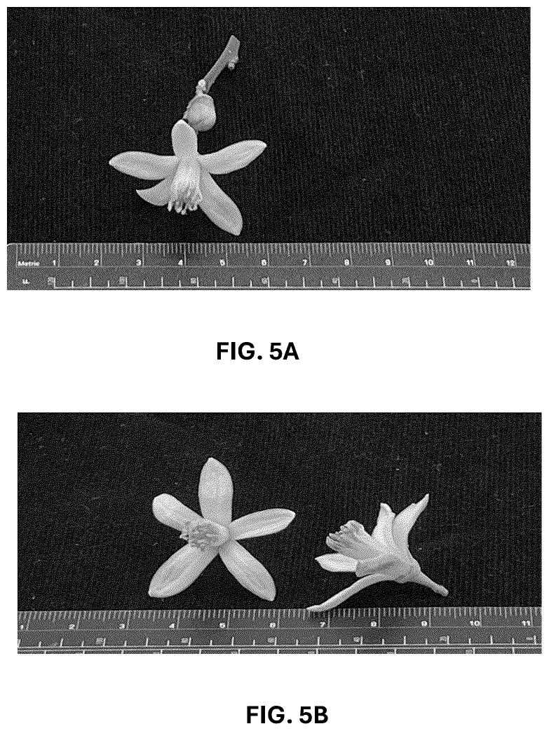

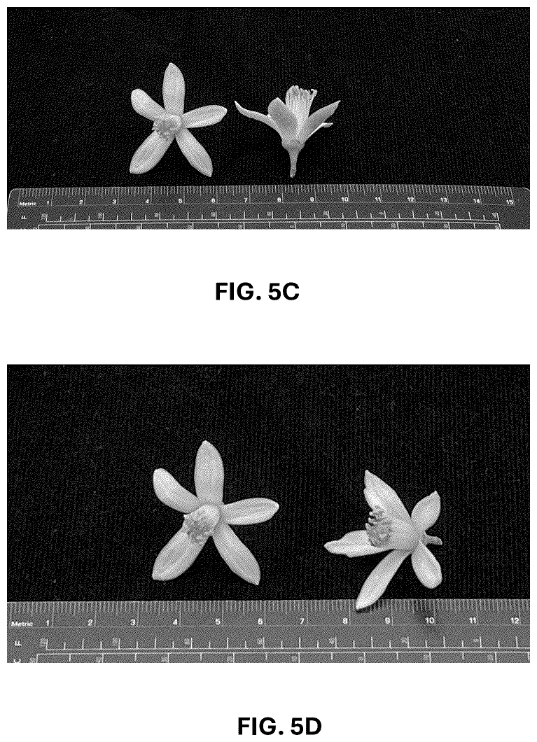

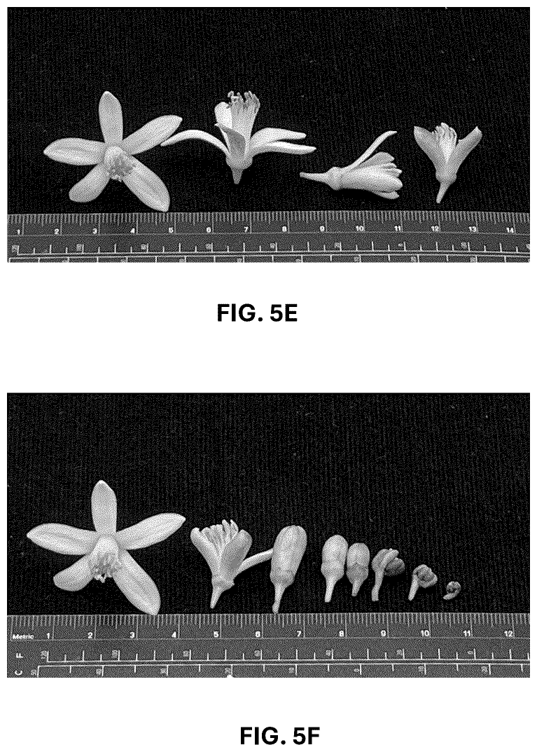

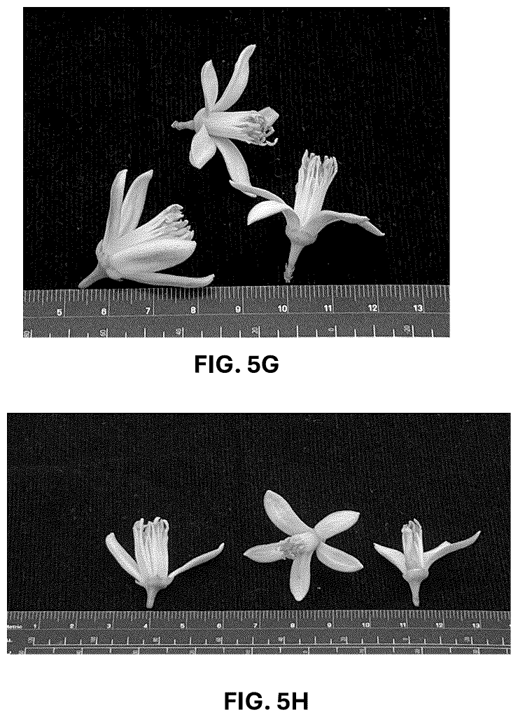

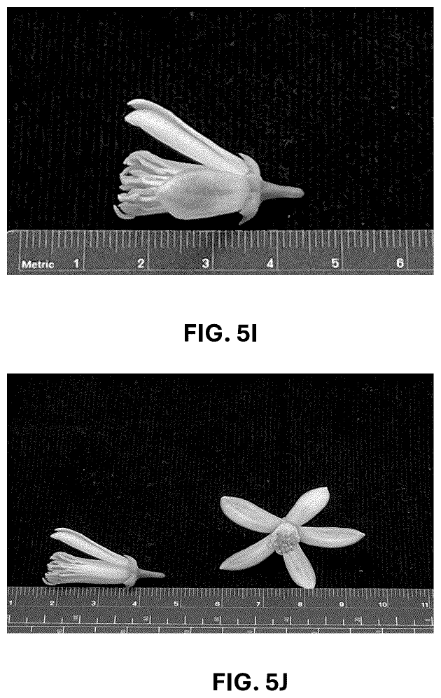

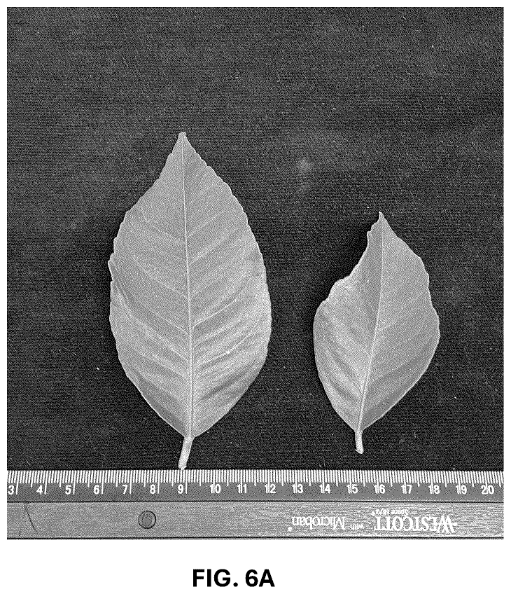

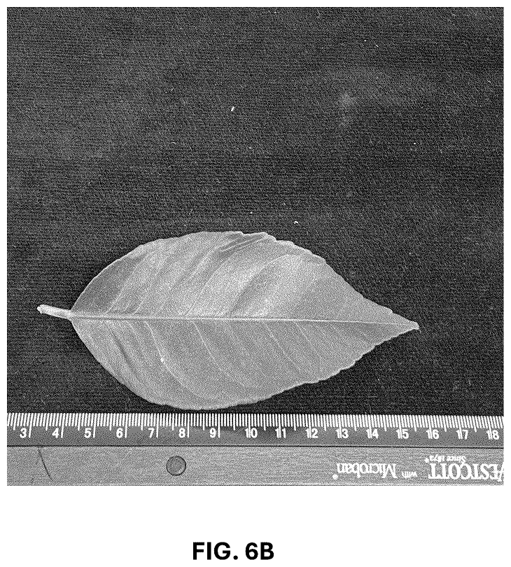

### DETAILED BOTANICAL DESCRIPTION OF THE CULTIVAR

In the following description, color references are made to The Royal Horticultural Society (R.H.S.) Colour Chart, 1986 Edition, except where general terms of ordinary dictionary significance are used.
Botanical description:
   Botanical classification.—Family: Rutaceae. Botanical name: Citrus limon (L.) Burm. Common name: Lemon. Cultivar: ‘CE-D5-1-9-42’.
   Parentage.—The new lemon variety ‘CE-D5-1-9-42’ arose as an irradiated budwood clone from ‘Cook Eureka’ lemon.
   Tree.—Ploidy: Diploid. Size: Medium compared to the tree age (young tree). Width across row: 1.5 meters on average. Width down row: 1.4 meters on average. Tree height: 2.1 meters on average. Tree canopy diameter: 1.7 meters on average. Vigor: Moderate. Density: Canopies are moderately dense. Tree shape (form): Oblate shape (obloid). Growth habit (current season): Vegetative growth in general is moderate to vigorous.
   Trunk.—Trunk diameter: Approximately 4.5 cm at 30 cm above the ground. Trunk texture: Medium rough. Trunk bark color: RHS N200B (Brown Group).
   Branches.—Branch length: 1.3 meters on average. Branch diameter: 1.8 cm on average. Crotch angel: 75-80 degrees on average (acute with an angle of less than 90 degrees). Branch texture: Relatively rough with long spines. Branch color: RHS 200D (Brown Group). Spine size: Approximately 3.5 cm in length and approximately 3.5 mm in diameter. Spine color: RHS N199A (greyed brown), with some being RHS 139B (green), depending on the branch age.
   Leaves.—Size (lamina average): Length: 110 mm on average. Width: 65 mm on average. L/W ratio: 1.7 on average. Thickness: Regular and average for lemon. Type: Simple (unifoliate). Shape: Elliptical. Apex: Acute to acuminate. Base: Acute to obtuse. Margin: Entire to undulate. Surface: Upper surface: Smooth. Lower surface: Medium veins that are pinnately netted. Color: Upper surface (adaxial): RHS N 137B (Green Group). Lower surface (abaxial): RHS 143C (Green Group). Petiole: Shape and attachment: Brevipetiolate; shorter than the leaf lamina. The junction between the petiole and the lamina is articulate. Shape (petiole wing): Obovate with narrow end at the base. Length: 15 mm on average. Width: 2.5 mm on average. Color: RHS 137B (Yellow-Green Group).
   Flowers.—Type: Perfect and staminate. Flower bearing: Single or cluster. If borne in clusters, each cluster consists of 2-5 flowers. If borne individually, individual flowers grow from leafy terminals and leaf axillaries. Flower diameter: Fully open flower has an average diameter of 32 mm. Flower depth: 15 mm on average. Flower blooming period: Trees have a major bloom season in central Florida that can begin in early January. However, trees will bloom sporadically throughout the year, depending on environmental influences (cold weather and drought will stimulate additional flowering). First bloom: January. Full bloom: February. Fragrance: Fragrant. Flower bud size: Length: Approximately 2 mm for the initial visible flower bud; approximately 22 mm for the mature flower buds before opening. Diameter: Approximately 2 mm in the initial visible flower bud; approximately 7 mm in the mature bud. Flower bud shape: Initial visible flower bud with round dome shape; mature flower bud with elongated olive shape. Flower bud color: RHS 186B (Greyed-Purple Group) for the initial visible flower bud; RHS 186D (Greyed-Purple Group) mixed with RHS NN 155B (white) for mature buds.
   Reproductive organs.—Petals: Number: 5. Petal length: 25 mm on average. Petal width: 8 mm on average. Petal shape: Flat spatula shape. Apex shape: Smooth with obtuse angle. Base shape: Obtuse. Petal Color: Upper Surface: RHS 186D (Greyed-Purple Group) mixed with RHS NN 155B (white). Lower surface: RHS NN 155B (White Group). Margin: Smooth. Sepals: Number (per flower): 5.Shape: Delta shaped with acute angle at apex, although some with flat angle. Length: 4 mm on average. Width: 3 mm on average. Apex shape: Acute angle at apex. Margin: Smooth. Color: Upper surface: RHS 157B (green-white) with RHS 186D (Greyed-Purple Group) areas. Lower surface: RHS 157A (green-white). Pedicel: Length: 10 mm on average. Diameter: 2 mm on average. Color: RHS 144B (Yellow Group). Stamen: Number: 32 per flower on average. Length: 20 mm on average.
   Anther.—Length: 4 mm on average. Width: 2 mm on average. Color: RHS 10A (yellow). Pollen color (general): RHS 11A (yellow). Pistil (in perfect flowers only): Number: 1. Length: 15 mm on average. Color: RHS 154D (yellow green). Style length: 16 mm on average. Style diameter: 2 mm on average. Style color: RHS 144A (yellow green). Ovary shape: Oval shape. Ovary diameter: 3 mm on average. Ovary color: RHS 142B (green).
   Fruit.—Size: Not necessarily uniform. In general, it is elongated oval to oval shaped with a neck that varies in size. Average weight (per individual fruit): 97 grams on average. Length: 7 cm on average. Diameter: 6 cm on average. Shape: Oval to elongated oval with elongated tip. Shape (cross-section): Round. Apex: Prominently bulged, nipple-like shape on many fruit. Base: Short neck. Harvest: Date of first pick: Late May to late July from main bloom. Date of last pick: Can be harvested periodically throughout the year, depending on juice percentage, peel color, etc. Fruit stem: Length: 15 mm on average. Diameter: 4.5 mm on average. Color: RHS 146A (yellow green).
   Rind.—Adherence: Albedo (mesocarp) to flesh (endocarp) is medium to strong. Thickness: Medium (4 mm on average). Texture: Firm with excellent flavor. Surface texture: Smooth to slightly bumpy. Color: Flavedo (epicarp): RHS 146B (Yellow-Green Group). Albedo (mesocarp): RHS NN155C (White Group). Style end: Closed. Rind oil cell density: 70-75 cell/square cm. Oil gland size: 1.5 mm on average.
   Flesh.—Number of segments: 8-9.Segment length: 6.5 cm on average. Segment width: 2.5 cm on average. Juice: Abundant. Color: RHS 146C (yellow green). Texture: Soft to medium. Vesicles: Medium thickness. Length: 10 mm on average. Diameter: 2.5 mm on average. Juice quality: Brix: 6.8. Acidity (average): 4.2%. Ratio: 1.6. Juice color: 29.
   Seeds.—Type: Seedless (observed seed count is 0-2 seeds per fruit, with the presence of seeds being rare). Seed shape, when found: Ovoid pointed. Fruit parthenocarpy: Yes.

What is claimed is:

1. A new and distinct lemon plant named ‘CE-D5-1-9-42’ as illustrated and described herein.
